(19) United States
(12) Patent Application Publication
(10) Pub. No.: US 2010/0179391 A1
(43) Pub. Date: Jul. 15, 2010

(54) SYSTEMS AND METHODS FOR A WIRELESS SENSOR PROXY WITH FEEDBACK CONTROL

(75) Inventors: Felix Clarence Quintanar, II, Boca Raton, FL (US); Randall L. Luck, Cary, NC (US); Gary D. Turner, Lilburn, GA (US); Mark Joseph Phelps, Atlanta, GA (US)

Correspondence Address: SUTHERLAND ASBILL & BRENNAN LLP, 999 PEACHTREE STREET, N.E., ATLANTA, GA, 30309, US

(73) Assignee: LIFESYNC CORPORATION, Fort Lauderdale, FL (US)

(21) Appl. No.: 12/354,295

(22) Filed: Jan. 15, 2009

(51) Int. Cl. A61B 5/00

(52) U.S. Cl. 600/301

(57) ABSTRACT

Systems and methods may be provided for wirelessly monitoring physiological vital signs. The systems and methods may include transmitting, from a local replication system via a wireless communications link, one or more stimulus signals to a remote signal acquisition subsystem that may be in communication with at least one remote sensor, where, responsive to the one or more stimulus signals, the at least one remote sensor is operable to generate one or more interrogation signals applied to a physiological system under test, where the at least one remote sensor may detect one or more response signal, where the one or more response signals may include a detected physiological system response to the one or more interrogation signals. The systems and methods may further include receiving, at the local replication system via the wireless communication link, the one or more response signals detected by the at least one remote sensor and transmitted from the remote signal acquisition subsystem, and where the one or more received response signals may be utilized as part of a feedback loop for controlling any subsequently transmitted stimulus signals.

### FIELD OF THE INVENTION

The present invention generally relates to monitoring patient vital signs, and more particularly, to a system and method for wirelessly monitoring patient vital signs using feedback control.

### BACKGROUND OF THE INVENTION

A vast majority of the vital-sign monitoring equipment in hospitals obtain physiological measurement information from sensors that are attached to a patient's body, and the sensors are typically connected to the monitor via a cable. A patient's mobility can be severely limited when they are tethered to monitoring equipment, and each dangling cable presents a potential tripping-, unplugging-, or tangling-hazard to the patient and the caregiver. To overcome this problem, wireless monitors have been developed. Examples of wireless systems for patient monitoring include U.S. Pat. Nos. 6,850,788 to Al-Ali, 6,289,238 to Besson et al., 6,731,962 to Katarow et al. and 6,954,664 to Sweitzer et al. Each of these example prior art references describe wireless systems that can eliminate the cable between a sensor and a monitor; however, none of the references describe systems or methods that can detect a physiological response to a stimulus when feedback is required to control the proper level of stimulus. For example, in the case of Al-Ali (U.S. Pat. No. 6,850,788), the sensor signal is derived at an independent remote measurement system, and is transmitted one-way to a local adaptation system that interfaces to the monitoring equipment. Similarly, Katarow et al (U.S. Pat. No. 6,731,962) and Sweitzer et al (U.S. Pat. No. 6,954,664) are limited to one-direction wireless communication of the measurement derived by the remote measurement system. For these systems, the absence of bi-direction wireless communication prevents transmission of sensor feedback from the measurement system to the remote sensor.

Therefore, bi-directional wireless communication is necessary to complete a feedback loop. Bi-directional communication may be necessary, hut may not be sufficient for adequately closing a feedback loop in a wireless link. For example, Besson et al (U.S. Pat. No. 6,289,238) uses a bi-directional wireless communication system, but the transmission from the base unit (evaluator station) to the remote sensor (electrode) is primarily used for setting-up and controlling the transmission parameters at the remote sensor to ensure efficient, reliable wireless link for the one-direction communication of non-specific sensor signals, with error correction. The wireless system of Besson; however, does not utilize feedback to control the sensor's stimulus level as a function of the measured response.

With properly designed system architecture and bi-directional communication, feedback control via a wireless link becomes possible. But the accuracy of a wirelessly monitored measurement may further depend upon prior knowledge of the sensor's characteristics, and therefore, calibration is an additional consideration. For example, in the case of pulse oximetry, calibration information is typically encoded in the sensor head using a resistor or other memory device to identify the calibration characteristic of red and IR light sources that are used for measuring the patient's blood-oxygen level.

Therefore, the need exists for a system and method that will facilitate wireless communication between a vital sign monitor and a sensor, where sensor information, feedback and calibration data can be handled transparently, as if the sensor were directly connected to the vital sign monitor with a cable.

### BRIEF SUMMARY OF THE INVENTION

According to an example embodiment of the invention, there may be a method of wirelessly monitoring physiological vital signs. The method may include transmitting, from a local replication system via a wireless communications link, one or more stimulus signals to a remote signal acquisition subsystem that may be in communication with at least one remote sensor, where, responsive, to the one or more stimulus signals, the at least one remote sensor may be operable to generate one or more interrogation signals applied to a physiological system under test, where the at least one remote sensor may detect one or more response signal, where the one or more response signals may include a detected physiological system response to the one or more interrogation signals. The method may further include receiving, at the local replication system via the wireless communication link, the one or more response signals detected by the at least one remote sensor and transmitted from the remote signal acquisition subsystem, and where the one or more received response signals may be utilized as part of a feedback loop for controlling any subsequently transmitted stimulus signals.

According to an example embodiment invention, there may be a system for wireless monitoring of physiological vital signs. The system may include a transceiver operable to transmit from a local replication system via a wireless communications link, one or more stimulus signals to a remote signal acquisition subsystem that may be in communication with at least one remote sensor, where the remote signal acquisition subsystem may include a transceiver operable to receive the stimulus signals from the local replication system via the wireless communication s link, and responsive, to the one or more stimulus signals, the at least one remote sensor is operable to generate one or more interrogation signals applied to a physiological system under test, where the at least one remote sensor may detect one or more response signal, where the one or more response signals may include a detected physiological system response to the one or more interrogation signals. The system may further include a transceiver operable to transmit from the remote signal acquisition subsystem via a wireless communications link, one or more response signals to the local replication subsystem where the transceiver at the local replication subsystem may be operable to receive, via the wireless communication link, the one or more response signals detected by the at least one remote sensor and transmitted from the remote signal acquisition subsystem; and where the one or more received response signals may be utilized as part of the feedback loop for controlling any subsequently transmitted stimulus signals. Embodiments of the invention may further provide a system and method for detecting and utilizing the calibration and/or identification data for a particular sensor.

According to an embodiment of the wireless sensor proxy with feedback control, the wireless system can be completely agnostic with respect to the type of measurement being performed, and therefore, the system may be utilized for wirelessly monitoring blood oxygen, blood pressure, blood carbon dioxide, respiration, etc. by pairing adaptors located at the local monitoring equipment and the remote sensor.

### BRIEF DESCRIPTION OF THE DRAWINGS

Reference will now be made to the accompanying drawings, which are not necessarily drawn to scale, and wherein:

FIG. 1 illustrates an example system for remote sensing with feedback using a wireless link, according to an example embodiment of the invention.

FIG. 2 is a flowchart of an example method for remote sensing using an example wireless link with feedback, according to an example embodiment of the invention.

FIG. 3 illustrates an example representation of a local replication subsystem, according to an example embodiment of the invention.

FIG. 4 illustrates an example representation of a remote signal acquisition subsystem, according to an example embodiment of the invention.

FIG. 5 illustrates an example pulse oximeter remote sensor, according to an example embodiment of the invention.

FIG. 6A is a flowchart of an example method for setting up the remote signal acquisition system to obtain calibration or identification information from the example remote sensor, and for communicating this information to the local replication system, according to an example embodiment of the invention.

FIG. 6B is a flowchart of an example method for setting up the replication of the calibration or identification information at the local replication system, including communicating the replicated calibration or identification information to the local measurement system, according to an example embodiment of the invention.

### DETAILED DESCRIPTION OF THE INVENTION

Embodiments of the invention will be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

An embodiment of the invention may enable wireless operation of a sensor system via a wireless proxy in place of a cable that would otherwise tether a sensor to a vital-sign monitor. The term “proxy” may mean substitute, stand-in, or replacement, according to an example embodiment of the invention. In eliminating the cable, the wireless proxy may transparently handle all of the necessary communication, including calibration setup, measurement, and feedback, as described herein.

In wired communication systems requiring feedback, voltages applied to a communication wire are transmitted from the source to the destination at nearly the speed of light, and therefore, signal round-trip time-delay, i.e., latency, is typically so small that feedback loop performance is not adversely impacted due to the latency. In contrast, time-delays in a wireless communication systems can be so significant that the achievable bandwidth of the feedback loop is reduced, thereby limiting the speed at which the communication system can remain under feedback control. Described herein are systems and methods that address the issues associated with latency in the wireless communication system, according to example embodiments of the invention.

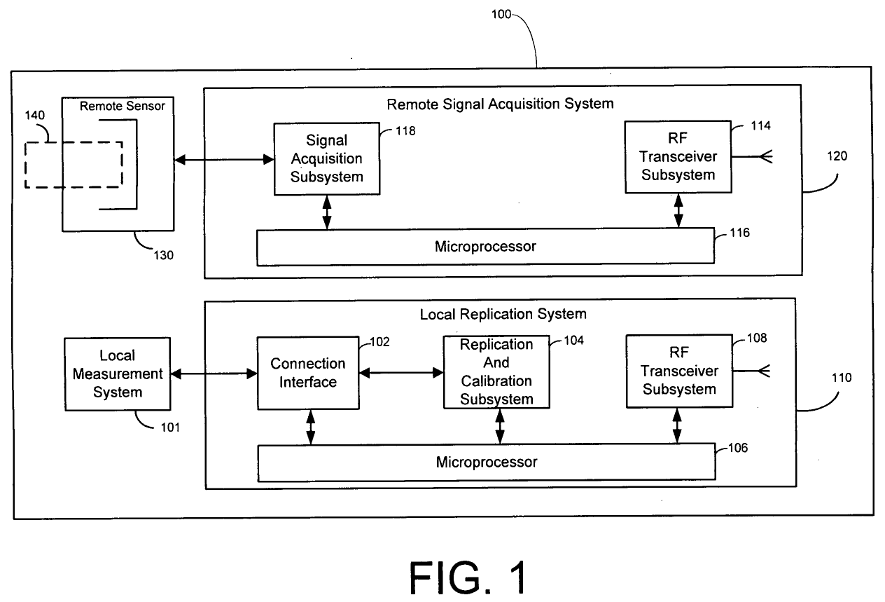

For the purpose of illustration, embodiments of the present invention will now be described in the context of the accompanying figures and flow diagrams, according to an embodiment of the invention. FIG. 1 illustrates an example system 100, that may include a local measurement system 101, a local replication system 110, a remote signal acquisition system 120, and a remote sensor 130. The local measurement system 101, which may comprise vital sign monitoring equipment, may be operative to produce stimulus signals, receive and process response signals, adjust the stimulus signals based on the received response signals, and receive calibration and/or identification data for processing and interpreting the response signals. The local replication system 110 may be operative to process, transfer, and transmit the stimulus signal from the local measurement system 101 to the remote signal acquisition system 120, to receive the response signal transmitted from the remote signal acquisition system 120, and to process and transfer the response signal to the local measurement system 101. The local replication system 110 may also be operative to replicate the remote sensor 130 calibration and/or identification information so that it can be communicated to the local measurement system 101. The remote signal acquisition system 120 may be operable to receive stimulus signals that are transmitted from the local replication system 110, and to process the stimulus signals and transfer them to the remote sensor 130. The remote signal acquisition system 120 may also be operable to transfer, process, and transmit response signals and calibration and/or identification data from the remote sensor 130 to the local replication system. The remote sensor 130 may be operable to interrogate a physiological system 140 with converted stimulus signals, and may detect corresponding response signals that result from the interrogation.

The local measurement system 101 may be electrically connected to the local replication system 110, and the remote signal acquisition system 120 may be electrically connected to the remote sensor 130. According to an example embodiment of the invention, the local replication system 110 may communicate wirelessly with the remote signal acquisition system 120.

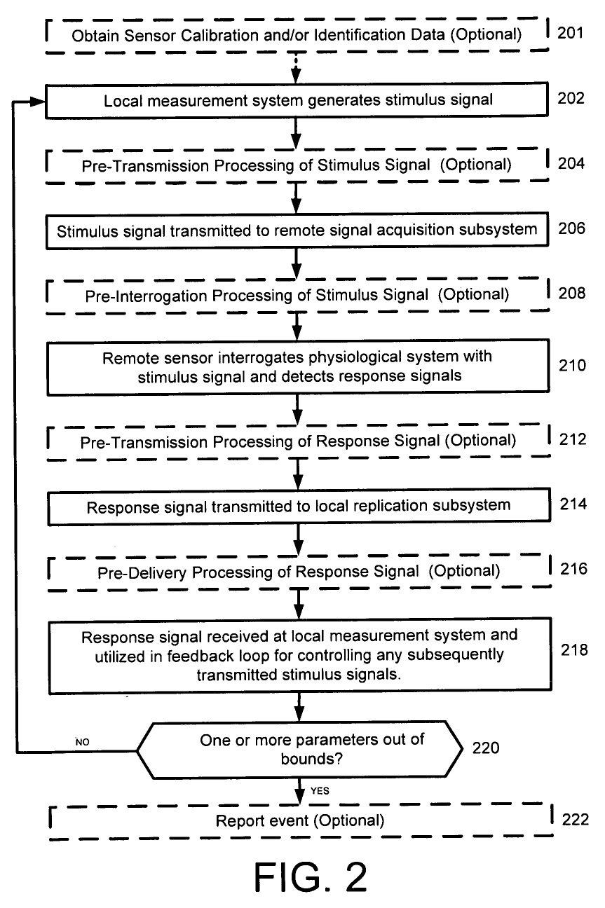

An example operation of the system 100 in FIG. 1 will now be further described using the example flowchart of FIG. 2. Beginning with block 201 of FIG. 2, the remote signal acquisition system 120 may read the remote sensor 130 calibration and/or identification data and wirelessly communicate the data to the local measurement system 101 via the local replication system 110. In an example embodiment, the calibration and/or identification data may be represented by a measurable analog element, such as a resistor. In another example embodiment, the calibration and/or identification data may be represented in a readable digital code and stored in a non-volatile memory within the remote sensor 130. Example details of this optional calibration procedure will be discussed in more detail in the CALIBRATION AND SETUP EXAMPLE section below. In block 202, the local measurement system 101 may generate one or more stimulus signals that may be operative to drive the remote sensor 130. The stimulus signals may include one or more electrical waveforms that may be utilized by the remote sensor 130 to generate one or more interrogation signals that are applied to the physiological system 140 under test. As an example, the remote sensor 130 may convert the stimulus signals to interrogation signals that may comprise one or more of light, electrical current, radiation, radio frequency, heat, vibration, or other forms of energy.

Still referring to FIG. 2, in block 204, the local replication system 110 may receive the stimulus signals generated by the local measurement system 101 through the connection interface 102. In block 204, the received stimulus signal may optionally undergo pre-transmission processing via the signal replication and calibration subsystem 104 and microprocessor 106 of the local replication system 110. As an example, pre-transmission processing may include one or more of: analog-to-digital conversion, level shifting, frequency shifting, phase shifting, and/or amplitude adjustment, according to an example embodiment of the invention. It will be appreciated that other pre-transmission processing may be available in accordance with an example embodiment of the invention. Following any optional pre-transmission processing in block 204, processing may proceed to block 206, where the RF transceiver subsystem 108 may wirelessly transmit the stimulus signals to the remote acquisition system 120. The remote acquisition system 120 may receive the transmitted stimulus signals via the RF transceiver subsystem 114. In block 208, the stimulus signals received at the remote signal acquisition system 120 may optionally undergo pre-interrogation processing via microprocessor 116 and signal acquisition subsystem 118. Example pre-interrogation processing in block 208 may include: digital-to-analog conversion, level shifting, frequency shifting, phase shifting, timing adjustment, and/or amplitude adjustment. It will be appreciated that other pre-interrogation processing may be available in accordance with an example embodiment of the invention.

In block 210 of FIG. 2, the remote signal acquisition system 120 may deliver the stimulus signals to the remote sensor 130. The remote sensor 130 may generate interrogation signals (e.g., light, electrical current, radiation, radio frequency, heat, vibration, indirect pressure, etc.) responsive to the received stimulus signals, and may simultaneously detect the corresponding response signals. The response signals may be attenuated and/or modulated versions of the interrogation signal. The response signals can result from the stimulus signal passing through, or otherwise, acting on part of a patients body. Likewise, the response signals may be representative of, or otherwise associated with, the physiological system 140 under test. In block 210, the response signals may be provided from the remote sensor 130 to the remote acquisition subsystem 120.

As indicated in block 212, the detected response signal may optionally undergo pre-transmission processing via the signal acquisition subsystem 118 and microprocessor 116 prior to being transmitted to the local replication system 110 via RF transceiver subsystems 114 and 108 as indicated in block 214. Block 216 indicates that the local replication system 110 may optionally perform pre-delivery processing (e.g., digital-to-analog conversion, level shifting, frequency shifting, phase shifting, amplitude shifting, time adjusting, etc.) prior to delivery of the response signals back to the local measurement system 101. In block 218, the response signal may be received by the local measurement system 101. The local measurement system 101 may use the response signal as part of a feedback loop for controlling any subsequently transmitted stimulus signals. For example, one or more parameters (e.g., amplitude, phase, etc.) of the stimulus signal may be adjusted based upon the received response signal. In blocks 220 and 222, the local replication system 110 may optionally report an event if any parameters are out of bounds. For example, if the round trip delay (also known as the latency) imposed by the system 100 approaches or exceeds the time constant of the feedback loop, such a condition may constitute an instability that may require further manual or automatic adjustments to the system, or may necessitate the sounding of an alarm, according to an example embodiment of the invention. Other events (e.g., absence of a remote sensor, RF transceiver signal fade, subsystem errors, etc.) may also be reported in block 222 without departing from example embodiments. It will be appreciated that many variations of FIGS. 1 and 2 are available without departing from example embodiments of the invention. For example, microprocessor 106 may be operative to process a portion or all of the functions of the connection interface 102 and the replication and calibration subsystem 104. Similarly, microprocessor 116 may be operative to process a portion of all of the functions of the signal acquisition subsystem 118.

According to an example embodiment of the invention, the response signal from the remote sensor 130 may be too strong or too weak for the local measurement system 101 circuitry. For example, if the response signal amplitude exceeds the dynamic range of the local measurement system 101 A/D converter, the measurement determined by the local measurement system 101 may be prone to overdrive errors. On the other hand, if the response signal is too weak, the measurement accuracy of the local measurement system 101 may suffer from excess noise. Therefore, by using the response signal as feedback, the local measurement system 101 may adjust the average amplitude level of the stimulus signal so that the response signal level may be optimized for accurate detection, according to an example embodiment of the invention.

According to an example embodiment of the invention, and as indicated above with respect to blocks 220 and 222 of FIG. 2, an alert can be reported in blocks 220 and 222 if one or more of the parameters of the communication system are out of normal bounds. For example, if the round-trip delay, or latency, imposed by the wireless proxy (e.g., the remote signal acquisition system 120 and the local replication system 110), were to exceed a pre-determined value, appropriate action can be taken, including producing an alert or alarm. The alert may be utilized internally by microprocessors 106, 116, associated circuitry, and firmware to adjust communication parameters (power, channel, protocol, etc) or the rate at which the amplitude of the stimulus signal varies, for example, so that the feedback loop is brought under control. If pre-programmed measures are not able to bring the parameters within pre-determined bounds, then the alarm may be utilized, for example, to notify hospital staff that the equipment has malfunctioned, that batteries need replacing, or that the patient has wandered outside of the range of the wireless communications channel, etc. It will be appreciated that many variations of alerts, alarm, and subsequent manual or automatic processes are available without departing from example embodiments of the invention.

According to an embodiment of the invention, the latency of the wireless communication loop may be monitored by periodically forming and transmitting data packets (with unique codes or digital time-stamps) from the local replication system 110 to the remote signal acquisition system 120, and back to the local replication system 110. The time stamp within the packet that has undergone the round-trip can be compared with the current time via microprocessor 106 to get an estimate of the latency. If the latency approaches or exceeds a predetermined value, an event can be reported and appropriate action can be taken, as mentioned in the preceding paragraph.

According to example embodiments of the invention, the wireless communication channel latency, as mentioned above, may be compared with a value representing the time constant, sample rate, or period of the stimulus signal requiring feedback control to determine if the system is operating properly. For example, a stimulus signal may contain relatively high frequency information (>1 KHz), but the feedback may only be required for control of the average, relatively slowly varying amplitude of the stimulus signal (<10 Hz). Therefore, in this example, the system could tolerate a latency up to 100 milliseconds.

### EXAMPLE EMBODIMENT

### Pulse Oximetry

It will be appreciated that FIGS. 1 and 2 may be applicable to a variety of healthcare applications, including pulse oximetry. In general, pulse oximetry may rely upon the absorption (or attenuation) of light as it transmits through a patient's tissue and blood. The light absorption may vary as a function of one or more of (1) the oxygen saturation level in the blood, (2) the wavelength of the light and (3) the thickness and optical density of the skin, cartilage, bone, tissue, etc. of the patient under test.

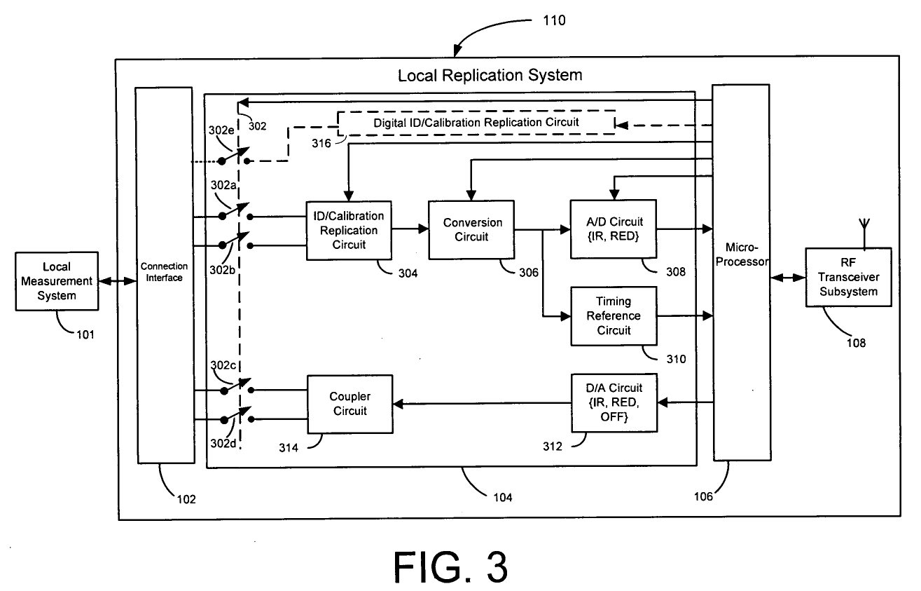

An example system embodiment suitable for pulse oximetry monitoring will now be described with reference to FIGS. 3-6. FIG. 3 depicts an example local replication system 110 that may be utilized for pulse oximetry, according to an embodiment of the invention. The example local replication system 110 may include a connection interface 102, a signal replication and calibration subsystem 104, a microprocessor 106, and a RF transceiver subsystem 108. According to an example embodiment of the invention, the connection interface 102, may provide a convenient connection to the local measurement system 101. The replication and calibration subsystem 104 may include switching network 302, under the control of microprocessor 106 for providing connections between the connection interface 102, the coupler circuit 314, and the ID/calibration replication circuit 304. The ID/calibration replication circuit 304 may be operable to emulate or replicate calibration or identification information, under control of microprocessor 106 for reading by the local measurement system 101, and will be explained in detail below in the CALIBRATION AND SETUP EXAMPLE. The replication and calibration subsystem 104 may also include conversion circuit 306 to provide, for example, current-to-voltage conversion, level shifting, amplification, filtering, etc. for the stimulus signal. The conversion circuit 306 may output the conditioned stimulus signal to the analog-to-digital conversion by circuit 308, where the stimulus signal may be further altered via microprocessor 106 prior to being transmitted to the remote signal acquisition system 120 via RF transceiver subsystem 108. The conditioned stimulus signal that is output from the conversion circuit 306 may also be utilized for extracting timing information via the timing reference circuit 310. Response signals received from the remote signal acquisition system 120 via RF transceiver subsystem 108 and microprocessor 106 may be converted to analog signals via digital-to-analog circuit 312. Analog response signals output from the D/A circuit 312 may be further conditioned (converted, filtered, level shifted, voltage-to-current (V/I) converted, etc.) by the coupler circuit 314 for appropriate reading by the local measurement system 101.

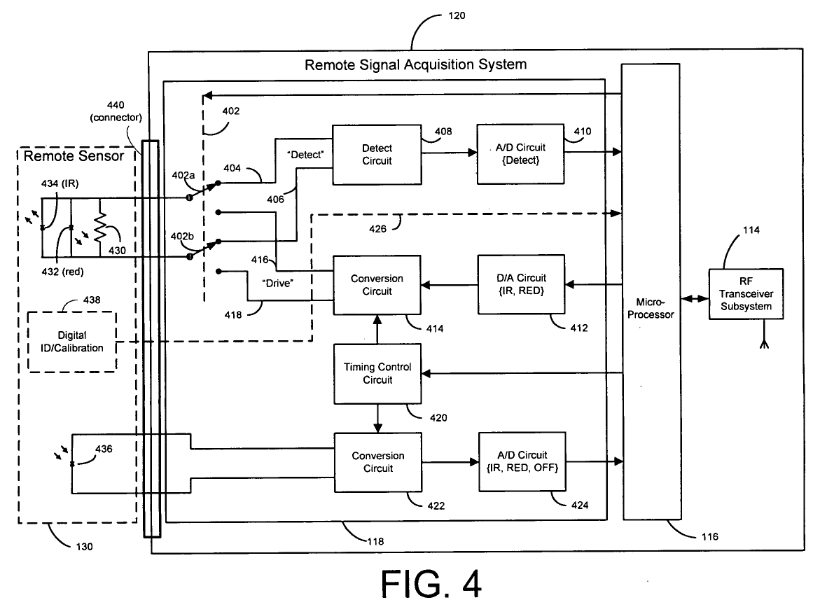

FIG. 4 depicts an example remote signal acquisition system 120 that may be utilized for pulse oximetry, according to an embodiment of the invention. The example remote signal acquisition system 120 may include a RF transceiver subsystem 114, a microprocessor 116, and a signal acquisition subsystem 118. Also shown in FIG. 4 is the schematic diagram of an example remote sensor 130. The remote signal acquisition system 120 is operable to receive stimulus signals from the local replication system 110 via the RF transceiver subsystem 114 and microprocessor 116. The stimulus signals may be converted from digital-to-analog by the D/A circuit 412 prior to being conditioned (amplified, time-shifted, level shifted, filtered, voltage-to-current converted, etc.) by the conversion circuit 414. The conversion circuit 414 may output a “Drive” signal via circuit traces 416 418, and connect to the remote sensor 130 via switch bank 402. The operation of the detect circuit 408 and the A/D circuit 410 will be covered in detail below in the CALIBRATION AND SETUP EXAMPLE. The remote sensor 130 may convert the stimulus “Drive” signal to an infra red (IR) and RED interrogation signal via LED's 434 432 for measuring the blood oxygen saturation of the patient under test. The patient's response to the interrogation signal can be detected at the photodiode 436. This response signal can be conditioned (amplified, time-shifted, level shifted, filtered, current-to-voltage converted, etc.) by conversion circuit 422 prior to being analog-to-digital (A/D) converted by A/D circuit 424, and transmitted to the local replication system 110 via microprocessor 116 and RF transceiver subsystem 114.

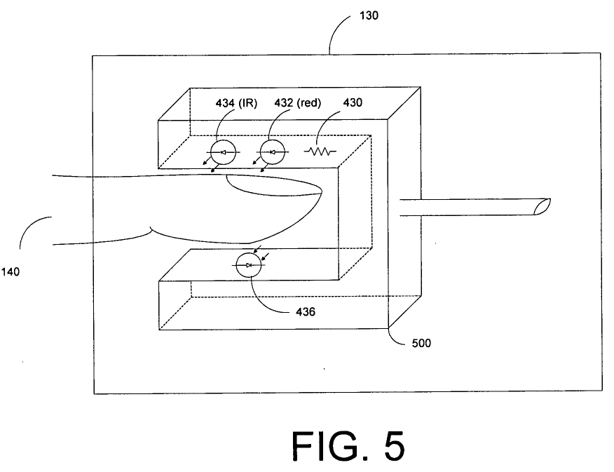

FIG. 5 depicts an example remote sensor 130 (e.g., pulse oximetry sensor) which may include a RED light emitting diode (LED) 432, an infra-red (IR) LED 434, a calibration/identification element 430, and a photodiode detector 436. The RED LED 432 may emit light having a peak emission wavelength around 660 nm, and the IR LED 434 may emit light having a peak emission wavelength around 940 nm. When physiological system 140 absorbing tissue, such as a finger, is placed between the LEDs 432 434 and the photodiode detector 436, the amount of light from each LED 432 434 transmitted through the intervening tissue may be detected by the photodiode 436. The ratio of the modulated component of the transmitted light from each LED 432 434 may be proportional to the oxygen saturation of the arterial blood in the capillary bed of the intervening tissue.

### Feedback Example

Since the thickness and optical density of a physiological system 140, such as a finger, may vary from patient to patient, and since only a small percentage of the stimulus light from the LEDs 432 434 may be transmitted through the finger and incident on the photodiode 436, feedback control may be employed to continuously adjust the average level of the interrogation signal (i.e., the light intensity from LEDs 432 434) so that the response level (i.e., the detected light at photodiode 436) may be optimized for accurate detection. To accomplish this task, the pulse oximeter (i.e., the local measurement system 101) may adjust a parameter of the transmitted stimulus signal, based upon the detected response of the photodiode 436, which may result in an adjustment of the relative optical power levels of the LED's 432, 434. This mechanism of adjusting the source optical power based upon the detector response may constitute a sensor feedback control loop.

### Calibration and Setup Examples

It should be appreciated that the sensor head 500, as illustrated in FIG. 5, may be prone to malfunction or may become damaged as a result of day-to-day use. Therefore, pulse oximetry monitors (e.g., local measurement system 101) may be designed to accommodate replacement sensor heads 500. As mentioned above, the absorption (or attenuation) of the light stimulus, as it transmits through the patient's tissue and blood, may vary as a function of one or more of (1) the oxygen saturation level in the blood, (2) the peak emission wavelength of the RED and IR LEDs, and (3) the thickness and optical density of the skin, cartilage, bone, tissue, etc. of the finger under test. Since a significant amount of variability is inherent in the LED manufacturing process, the peak emission wavelength of a LED can vary from sensor-to-sensor. Without prior knowledge of the unique characteristics for a particular sensor head, the measurement results, as processed by the pulse oximetry monitor, may be prone to variations or errors. Therefore, pulse oximeter sensors may encode calibration or identification data, perhaps within each sensor head 500, to identify, for example, characteristic of the LED pair such as peak emission wavelengths, relative optical power emitted by each LED for a given input current, and/or the model and serial number of the sensor head 500. In an example embodiment of the invention, each sensor head 500 may include an analog memory element (e.g., calibration/identification element 430) for storing calibration or identification data in an analog format. In an alternative example embodiment of the invention, each sensor head 500 may include digital memory element (e.g., digital ID/calibration element 438) for storing calibration or identification data in a digital format.

According to an example embodiment of the invention, the signal acquisition subsystem 118 and the replication and calibration subsystem 104 are operative to communicate calibration information from the remote sensor head 500 to the local measurement system 101. Example methods and systems for communicating the calibration information from the remote sensor head 500 to the local measurement system 101 can be grouped into one or more embodiments depending upon the form of the calibration and/or identification element. For example, in one embodiment, the calibration/identification element 430 within the remote sensor head 500 may be an analog device (for example, a resistor). In another example embodiment, the calibration/identification element 438 may be a digital device (for example, an electronic integrated circuit with non-volatile memory) and may be capable of storing and communicating a pre-programmed digital code via a serial interface (e.g., via I2C, SPI, Dallas 1 wire, Johnson counter, RS232, etc.). In each of the example embodiments below, an alternative embodiment is presented to account for both analog 430 and/or digital 438 calibration/identification elements.

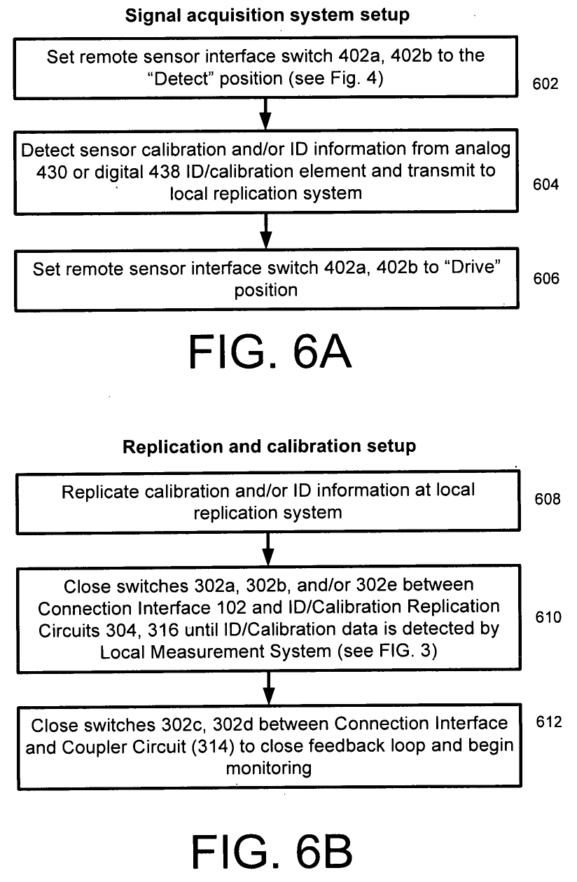

An example process for the signal acquisition system setup is depicted in the flowchart of FIG. 6A. The replication and calibration setup procedure is depicted in the flowchart of FIG. 6B. In block 602 of FIG. 6A, and with reference to FIG. 4, connector 440 may be utilized to connect the remote sensor 130 and the remote signal acquisition system 120. The sensor interface switch 402 may be set to the “Detect” position (e.g., switch 402a may be connected to path 404, and switch 402b may be connected to path 406), thereby allowing the detect circuit 408 to read analog calibration or identification information from calibration/identification element 430 of the remote sensor 130, as illustrated by block 604. The analog calibration or identification information may be read from calibration/identification element 430 by sourcing a known current through calibration/identification element 430, by measuring the voltage drop across the element (taking care to avoid forward biasing LEDs 432, 434), and by calculating the resistance as the voltage drop divided by the sourced current. The measured calibration or identification information (voltages and/or currents) may be provided to the A/D circuit 410 for converting analog calibration/ID information to digital calibration/ID information, and for calculation by the microprocessor 116. Alternatively, in an example embodiment of the invention, and in the case where the remote sensor 130 contains a digital identification/calibration element 438, the microprocessor 116 may directly read the calibration or identification information from the digital identification/calibration element 438 of the remote sensor 130 via optional circuit path 426. In either of the embodiments described above, the microprocessor 116 may receive the calibration or identification information and following any processing, may wirelessly transmit the calibration or identification information to the local replication system 110 via ° F. transceiver subsystem 114.

In block 606, once the calibration or identification information is obtained, the sensor interface switch 402 can be connected to the “Drive” position (e.g., switch 402a may be connected to circuit path 416 and switch 402b may be connected to circuit path 418) to enable driving LEDs 432, 434 with the appropriate stimulus signals for monitoring. Example processes for obtaining the sensor calibration or identification information have been described above with reference to the flowchart of FIG. 6A. The flowchart of FIG. 6B will now be utilized to describe example processes for communicating the calibration or identification information to the local measurement system 101 via the local replication system 110.

In block 608 of FIG. 6B, and with reference to FIG. 3, the calibration or identification information (read from the calibration/identification element 430 or from digital ID/calibration element 438) may be transmitted by the remote signal acquisition system 120 and stored, reproduced, or replicated at the local replication system 110 for reading by the local measurement system 101. Prior to receiving the remote sensor calibration/ID information, switch connections 302a through 302e may be in an open state, for example, to suspend operations of the local measurement system 101 while it waits for the introduction of calibration information. In one example embodiment, on receipt of analog calibration or identification information, microprocessor 106 may program the ID/calibration replication circuit 304 to the equivalent value of calibration/identification element 430. The microprocessor may further adjust the gain of the conversion circuit 306 to account for the value of the replicated calibration/identification element 430. For example, in one embodiment where the calibration/identification element 430 is analog (i.e., a resistor) the ID/calibration replication circuit 304, under control of microprocessor 106, may replicate (i.e., emulate, reproduce, etc.) the calibration/identification element 430 for reading by the local measurement system 101. The replication of the analog calibration/identification element 430 may be realized within the ID/calibration replication circuit 304 by utilizing a digital potentiometer, or similar variable resistance element. In an alternative embodiment where the remote sensor's 130 calibration/identification element is a digital identification/calibration element 438, (i.e., an integrated circuit with non-volatile memory), the replication (i.e., emulation, reproduction, etc.) of the digital code for reading by the local measurement system 101 may be realized by the microprocessor 106 alone or in combination with a digital ID/calibration replication circuit 316. In block 610, the replicated digital ID/calibration code may be presented to the local measurement system 101 via optional switch 302e using serial communication (e.g., via I2C, SPI, Dallas 1 wire, Johnson counter, RS232, etc.).

In block 610, and in an example embodiment where the calibration/identification element 430 is analog, the replicated calibration or identification information can read by local measurement system 101 by closing switch connections 302a and 302b of FIG. 3 to connect the connection interface 102 with the ID/Calibration replication circuit 304. In an example embodiment where the calibration/identification element 438 is digital, the replicated calibration or identification information can read by local measurement system 101 by closing switch connection 302e of FIG. 3 to connect the connection interface 102 with the digital ID/Calibration replication circuit 316. The calibration or identification information may be utilized by the local measurement system 101 to process the results of the physiological measurements. To prepare for measurements, as indicated in block 612, switch connections 302c and 302d can be closed to connect the connection interface 102 with the coupler circuit 314 for monitoring. The details of monitoring are further described in the following sections.

### Stimulus Signal Flow Example

Once the local measurement system 101 has completed calibration, it may generate the stimulus signal, which may be received by the local replication system 110 via connection interface 102. The ID/calibration replication circuit 304 may pass the stimulus signal to the conversion circuit 306, which may perform current-to-voltage (I-to-V) or voltage-to-voltage (V-to-V) conversion, and to the A/D conversion circuit 308 under control of microprocessor 106. The timing of the stimulus signals may be acquired by timing reference circuit 310 for further processing. The stimulus signal timing may include duty cycle, period and sequence for each of the remote sensor LED signals, i.e., RED LED 432 ON state, the IR LED 434 ON state and the OFF state. According to an embodiment of the invention, the stimulus and timing signals may be wirelessly transmitted to the remote signal acquisition system 120 by RF transceiver subsystem 108 under control of microprocessor 106.

According to an embodiment of the invention, and with reference to FIG. 4, the stimulus signals received at RF transceiver subsystem 114 are passed to microprocessor 116, and may be converted by D/A circuit 412 and conversion circuit 414, under control of timing control circuit 420, to provide the drive signal for the LEDs. At this point, switch paths 402a and 402b may already be connected to the “Drive” path, or conversion circuit 414. Therefore, the stimulus signal may provide the drive for the RED LED 432 and the IR LED 434.

### Response Signal Flow Example

With reference to FIG. 4, and according to an embodiment of the invention, the interrogation light radiation (as derived from the stimulus signals) from the RED LED 432 and the IR LED 434 may transmit through the finger of the patient, and an attenuated version of the interrogation light radiation may be incident upon detector 436, thereby producing a response signal. According to an example embodiment of the invention, the response signal may comprise small current signals that may require conversion to voltage signals by conversion circuit 422 under control of timing circuit 420. Conversion circuit 422 may output the response signal to analog-to-digital circuit 424 which may pass the digitized response signals to microprocessor 116.

According to an embodiment of the invention, the response signals may then be transmitted via RF transceiver 114 under control of microprocessor 116 to the local replication system 110 via RF transceiver 108 under the control of microprocessor 106. Referring now to FIG. 3, microprocessor 106, with input from the timing reference circuit 310, may multiplex and adjust the relative response signal timing before passing the response signal to D/A circuit (IR, RED, OFF) 312. The analog output from D/A circuit 312 may pass through coupler circuit 314 to the local measurement system 101 via the connector interface 102. In one embodiment of the invention, coupler circuit 314 may be a linear opto-coupler, however, it will be appreciated that other coupler circuits may be utilized as well, including non-linear opto-coupler circuits. According to one embodiment, the local measurement system not only utilizes the response signal for calculating the blood oxygen level, but it may also utilize a portion of the response signal for feedback in controlling the subsequent stimulus signals, thereby completing the feedback loop.

In an embodiment of the invention, the connector interface 102 can include an active replica of the physiological system 140 under test. For example, a material in the shape of a finger, with similar optical characteristics, that may modulate optical absorption based upon the control of the received response signal at the local replication subsystem 110. This embodiment may eliminate the need to design and manufacture custom connector interfaces 102 for each manufacturer's pulse oximetry system.

In an embodiment of the invention, any or all of the systems 101 110 120 130 or associated subsystems 102 104 108 114 118 may be powered by battery, by inductive coupling, by harvesting energy, or by a combination of power supplies including but not limited to rechargeable batteries, alternating current sources from standard wall plugs, direct current sources from dedicated power supplies, etc. Example methods that may be utilized for harvesting energy include piezoelectric, pyroelectric, electrostatic, thermoelectric, electrostatic, and ambient-radiation energy harvesting. Example devices for harvesting energy include electroactive polymers, variable capacitors, thermocouples, ferroelectric crystals, and solar cells.

According to an embodiment of the wireless sensor proxy with feedback control, the wireless system can be completely agnostic with respect to the type of measurement being performed, and therefore, the system may be utilized for wirelessly monitoring blood oxygen, blood pressure, blood carbon dioxide, respiration, etc. by adding interchangeable adaptors to the local monitoring equipment and the remote sensor.

Although the method and system is described herein with respect to wireless digital communications, one of ordinary skill in the art will recognize that other forms of wireless communications may be more advantageous for remote sensors dependent upon feedback control. Since the communications latency of analog wireless communications may be much less than that for digital wireless communications, examples of alternate methods of wireless communications include analog RF and light wave carrier. Furthermore, continuous methods of digital wireless communication, such as Frequency-Shift Keying (FSK) or Amplitude-Shift Keying (ASK), could have much less latency than packet-based digital transmission methods, such as Bluetooth or IEEE 802.11.

Although the method and system is described herein with respect to a pulse oximeter, one of ordinary skill in the art will recognize that the system and method may be adapted for any remote sensor that affects the desired measurement dependent upon feedback control from the local measurement system. Examples of sensors for which the current system and method may be adopted include non-invasive blood pressure sensors, blood carbon monoxide sensors, blood sugar sensors, side-stream capnography sensors, etc.

Although the example embodiments depicted in the figures and described herein includes one feedback channel, it is to be understood that the invention is not limited to the number of channels indicated in the example embodiments, but rather, the invention may comprise one or more measurement channels, and one or more feedback channels as needed by the end-use application.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method of wirelessly monitoring physiological vital signs, comprising:
transmitting, from a local replication system via a wireless communications link, one or more stimulus signals to a remote signal acquisition subsystem that is in communication with at least one remote sensors wherein
responsive, to the one or more stimulus signals, the at least one remote sensor is operable to generate one or more interrogation signals applied to a physiological system under test, wherein
the at least one remote sensor detects one or more response signal, wherein
the one or more response signals comprise a detected physiological system response to the one or more interrogation signals,
receiving, at the local replication system via the wireless communication link, the one or more response signals detected by the at least one remote sensor and transmitted from the remote signal acquisition subsystem, wherein the one or more received response signals are utilized as part of a feedback loop for controlling any subsequently transmitted stimulus signals.

2. The method of claim 1, wherein the wireless communication link comprises a digital or analog link.

3. The method of claim 1, wherein the transmission and reception of the wireless communication link are operative with one or more of
(a) light waves;
(b) radio frequency waves;
(c) inductive coupling; or
(d) capacitive coupling.

4. The method of claim 1, wherein the feedback loop for controlling the one or more stimulus signals utilizes the wireless communications link.

5. The method of claim 1, wherein the one or more received response signals are utilized for feedback in controlling the one or more stimulus signals.

6. The method of claim 1, wherein an event error code is reported if the communication link latency exceeds the time constant of the feedback loop or if any system parameters are out of pre-defined bounds.

7. The method of claim 1, wherein one or more calibration or identification signals received at the local replication system are replicated for communication with the local measurement system.

8. The method of claim 1, wherein the remote signal acquisition subsystem receives power via alternating line current, battery, inductive coupling, or by harvesting energy.

9. The method of claim 1, wherein the one or more received response or calibration signals at the local replication system are converted via an active replica of the physiological system under test and are in communication with the local measurement system.

10. The method of claim 1, wherein the remote sensor and the local monitoring system are operative with one or more of:
(a) pulse oximetry monitoring;
(b) respiration monitoring;
(c) side stream capnography monitoring;
(d) blood sugar monitoring;
(e) blood carbon monoxide monitoring; or
(f) blood-pressure monitoring.

11. A system for wirelessly monitoring physiological vital signs, comprising:
a transceiver operable to transmit from a local replication system via a wireless communications link, one or more stimulus signals to a remote signal acquisition subsystem that is in communication with at least one remote sensor, wherein
the remote signal acquisition subsystem comprises a transceiver operable to receive the stimulus signals from the local replication system via the wireless communication s link,
responsive, to the one or more stimulus signals, the at least one remote sensor is operable to generate one or more interrogation signals applied to a physiological system under test, wherein
the at least one remote sensor detects one or more response signal, wherein
the one or more response signals comprise a detected physiological system response to the one or more interrogation signals,
a transceiver operable to transmit from the remote signal acquisition subsystem via a wireless communications link, one or more response signals to the local replication subsystem, wherein
the transceiver at the local replication subsystem is operable to receive, via the wireless communication link, the one or more response signals detected by the at least one remote sensor and transmitted from the remote signal acquisition subsystem, wherein the one or more received response signals are utilized as part of a feedback loop for controlling any subsequently transmitted stimulus signals.

12. The system of claim 11, wherein the wireless communication link comprises a digital or analog link.

13. The system of claim 11, wherein the transceivers operative for wireless communication with one or more of:
(a) light waves;
(b) radio frequency waves;
(c) inductive coupling; or
(d) capacitive coupling.

14. The system of claim 11, wherein the feedback loop for controlling the one or more stimulus signals utilizes the wireless communications link.

15. The system of claim 11, wherein the one or more received response signals are utilized for feedback in controlling the one or more stimulus signals.

16. The system of claim 11, wherein an event error code is reported if the communication link latency exceeds the time constant of the feedback loop or if any of the system parameters are out of pre-defined bounds.

17. The system of claim 11 wherein the remote signal acquisition subsystem receives power via alternating line current, battery, inductive coupling, or by harvesting energy.

18. The system of claim 11 wherein the one or more received response signals at the local replication system are converted via an active replica of the physiological system under test and are in communication with the local measurement system.

19. The system of claim 11, wherein the remote sensor and the local monitoring system are operative with one or more of:
(a) pulse oximetry monitoring;
(b) respiration monitoring;
(c) side stream capnography monitoring;
(d) blood sugar monitoring;
(e) blood carbon monoxide monitoring; or
(f) blood-pressure monitoring.

20. The system of claim 11 wherein calibration information read from the remote sensor is transmitted to the local replication system, and wherein the local replication system replicates the calibration information for reading by the local measurement system.
